SEQUENCE LISTING (6 sequences)

SEQ ID NO: 1 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 2 - protein, 300 aa, Thermus aquaticus
MEENKRKSLENALKTIEKEFGKGAVMRLGEMPKLQVDVIPTGSLGLDLALGIGGIPRGRVTEIFGPESGGKTTLALTIIAQAQKGGGVAAFVDAEHALDPLYAKKLGVDVQELLVSQPDTGEQALEIVELLARSGAVDVIVVDSVAALVPKAEIEGEMGDQHVGLQARLMSQALRKLTAVLSKSNTAAIFINQVREKVGVMYGNPETTPGGRALKFYSSVRLDVRKSGQPIKVGNEAVGIKVKVKVVKNKLAPPFREAELEIYFGRGLDPVMDLVNVAVAAGVIEKAGSWFSYGEHRLGQ

SEQ ID NO: 3 - protein, 242 aa, Escherichia coli
MEGWQRAFVLHSRPWSETSLMLDVFTEESGRVRLVAKGARSKRSTLKGALQPFTPLLLRFGGRGEVKTLRSAEAVSLALPLSGITLYSGLYINELLSRVLEYETRFSELFFDYLHCIQSLAGVTGTPEPALRRFELALLGHLGYGVNFTHCAGSGEPVDDTMTYRYREEKGFIASVVIDNKTFTGRQLKALNAREFPDADTLRAAKRFTRMALKPYLGGKPLKSRELFRQFMPKRTVKTHYE

SEQ ID NO: 4 - protein, 342 aa, Homo sapiens
GSHMAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 5 - protein, 418 aa, Homo sapiens
MSGTEEAILGGRDSHPAAGGGSVLCFGQCQYTAEEYQAIQKALRQRLGPEYISSRMAGGGQKVCYIEGHRVINLANEMFGYNGWAHSITQQNVDFVDLNNGKFYVGVCAFVRVQLKDGSYHEDVGYGVSEGLKSKALSLEKARKEAVTDGLKRALRSFGNALGNCILDKDYLRSLNKLPRQLPLEVDLTKAKRQDLEPSVEEARYNSCRPNMALGHPQLQQVTSPSRPSHAVIPADQDCSSRSLSSSAVESEATHQRKLRQKQLQQQFRERMEKQQVRVSTPSAEKSEAAPPAPPVTHSTPVTVSEPLLEKDFLAGVTQELIKTLEDNSEKWAVTPDAGDGVVKPSSRADPAQTSDTLALNNQMVTQNRTPHSVCHQKPQAKSGSWDLQTYSADQRTTGNWESHRKSQDMKKRKYDPS

SEQ ID NO: 6 - protein, 391 aa, unidentified
MSDLKSRLIKASTSKLTAELTASKFFNEKDVVRTKIPMMNIALSGEITGGMQSGLLILAGPSKSFKSNFGLTMVSSYMRQYPDAVCLFYDSEFGITPAYLRSMGVDPERVIHTPVQSLEQLRIDMVNQLDAIERGEKVVVFIDSLGNLASKKETEDALNEKVVSDMTRAKTMKSLFRIVTPYFSTKNIPCIAINHTYETQEMFSKTVMGGGTGPMYSADTVFIIGKRQIKDGSDLQGYQFVLNVEKSRTVKEKSKFFIDVKFDGGIDPYSGLLDMALELGFVVKPKNGWYAREFLDEETGEMIREEKSWRAKDTNCTTFWGPLFKHQPFRDAIKRAYQLGAIDSNEIVEAEVDELINSKVEKFKSPESKSKSAADLETDLEQLSDMEEFNE

(19) United States
(12) Patent Application Publication
(10) Pub. No.: US 2023/0416805 A1
(43) Pub. Date: Dec. 28, 2023

(54) USE OF HOMOLOGOUS RECOMBINASE TO IMPROVE EFFICIENCY AND SENSITIVITY OF SINGLE CELL ASSAYS

(71) Applicant: Bio-Rad Laboratories, Inc., Hercules, CA (US)

(72) Inventors: Rahul RATTAN, Brighton, MI (US); Marta GONZALEZ-PLASKY, Grosse Pointe Woods, MI (US)

(21) Appl. No.: 18/209,350

(22) Filed: Jun. 13, 2023

Related U.S. Application Data
(60) Provisional application No. 63/352,975, filed on Jun. 16, 2022.

(51) Int. Cl. C12Q 1/6816

(52) U.S. Cl. CPC C12Q 1/6816

(57) ABSTRACT

Methods of decreasing bead aggregation and improving specificity of nucleic acid hybridization using non-specific nucleic acid binding proteins is described.

### CROSS-REFERENCE TO RELATED PATENT APPLICATIONS

The present application claims benefit of priority to U.S. Provisional Patent Application No. 63/352,975, filed Jun. 16, 2022, which is incorporated by reference for all purposes.

### SEQUENCE LISTING

The instant application contains a Sequence Listing which has been submitted electronically in XML format and is hereby incorporated by reference in its entirety. Said XML copy, created on Sep. 18, 2023, is named 094868-1389120_119510US_SL.xml and is 8,265 bytes in size.

### BACKGROUND OF THE INVENTION

Efficient capture of RNA, conversion into cDNA, and production of sequencing libraries is involved for achieving high sensitivity within single cell RNA applications. The assay input of a single cell has relatively few transcripts available for capture, so a loss of efficiency in any one of these steps results in loss of gene coverage and diversity. For example, several low-transcript genes have as few as 5-12 copies per cell.

### BRIEF SUMMARY OF THE INVENTION

In some embodiments, a method of introducing oligonucleotide-linked beads into partitions is provided. In some embodiments, the method comprises
providing a bulk solution comprising (i) a plurality of the oligonucleotide-linked beads and (ii) an amount of a non-sequence specific nucleic acid binding protein sufficient to reduce aggregation by the beads; and
introducing the beads into the partitions. In some embodiments, the number of beads in the partitions averages less than 1, 2, 3, 4, or 5 beads per partition.

In some embodiments, the partitions are microwells or droplets.

In some embodiments, at least a majority of the oligonucleotides coated on a bead have identical sequences and the oligonucleotides on different beads can be distinguished by a barcode sequence on the oligonucleotide.

In some embodiments, the oligonucleotides comprise a free 3′ end that comprises at least 3, 4, 5, or 6 contiguous thymine nucleotides that form a poly-T sequence.

In some embodiments, the partitions comprise single cells per partition during the introducing. In some embodiments, following the introducing, lysing the cells in the partitions and hybridizing the oligonucleotides in the partitions to target nucleic acids from the cells. In some embodiments, the method further comprises, before the hybridizing, adding a further amount of the non-sequence specific nucleic acid binding protein to the partitions. In some embodiments, the oligonucleotides are linked to the beads prior to the hybridizing. In some embodiments, the oligonucleotides are released from the beads prior to the hybridizing. In some embodiments, the method further comprises extending the oligonucleotides with a polymerase in a template-specific manner to link a reverse complement of the target nucleic acids to the oligonucleotides.

In some embodiments, the nucleic acids are RNA. In some embodiments, the nucleic acids are DNA.

In some embodiments, the non-sequence specific nucleic acid binding protein is selected from the group consisting of RecA, RecO, RecN, RadA, Rad51, Rad52 and UvsX.

Also provided is a solution comprising (i) a plurality of the oligonucleotide-linked beads and (ii) an amount of a non-sequence specific nucleic acid binding protein sufficient to reduce aggregation by the beads. In some embodiments, the oligonucleotides comprise a free 3′ end that comprises at least 6 contiguous thymine nucleotides that form a poly-T sequence In some embodiments, the non-sequence specific nucleic acid binding protein is selected from the group consisting of RecA, RecO, RecN, RadA, Rad51, Rad52 and UvsX.

Also provided is a method of introducing oligonucleotide-linked beads into partitions. In some embodiments, the method comprises providing a plurality of partitions, wherein the partitions comprise (i) an oligonucleotide-linked bead and (ii) a non-sequence specific nucleic acid binding protein, and (iii) a target nucleic acid; and hybridizing the oligonucleotide to the target nucleic acid in the presence of the non-sequence specific nucleic acid binding protein.

In some embodiments, the number of beads in the partitions averages less than 1, 2, 3, 4 or 5 beads per partition.

In some embodiments, the method comprises releasing the oligonucleotides from the bead prior to the hybridizing.

In some embodiments, the oligonucleotides comprise a free 3′ end that comprises at least 3, 4, 5, or 6 contiguous thymine nucleotides that form a poly-T sequence.

In some embodiments, at least a majority of the oligonucleotides linked to a bead have identical sequences and the oligonucleotides on different beads can be distinguished by a barcode sequence on the oligonucleotide.

In some embodiments, the partitions are microwells or droplets.

In some embodiments, the non-sequence specific nucleic acid binding protein is selected from the group consisting of RecA, RecO, RecN, RadA, Rad51, Rad52 and UvsX.

Also provided is a reaction mixture comprising (i) an oligonucleotide-linked bead and (ii) a non-sequence specific nucleic acid binding protein, and (iii) a target nucleic acid.

In some embodiments, the non-sequence specific nucleic acid binding protein is selected from the group consisting of RecA, RecO, RecN, RadA, Rad51, Rad52 and UvsX.

Also provided is a plurality of partitions, wherein the partitions contain a reaction mixture as described above or elsewhere herein, wherein at least a majority of the oligonucleotides linked to a bead in a partition have identical sequences and the oligonucleotides on different beads in different partitions can be distinguished by a barcode sequence on the oligonucleotide. In some embodiments, the partitions are microwells or droplets.

Also provided is a method of detecting a nucleic acid in a microfluidic channel. In some embodiments, the method comprises flowing a nucleic acid sample in a microfluidic channel past an immobilized oligonucleotide-linked bead in the presence of a non-sequence specific nucleic acid binding protein under conditions sufficient to allow for specific hybridization of the oligonucleotide to a target nucleic acid in the nucleic acid sample; and detecting hybridization of the target nucleic acid to the oligonucleotide. In some embodiments, the oligonucleotide-linked bead is immobilized in the microfluidic channel by optical tweezers.

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a bead” includes a plurality of such beads and reference to “the sequence” includes reference to one or more sequences known to those skilled in the art, and so forth.

An “oligonucleotide” is a polynucleotide. Generally oligonucleotides will have fewer than 250 nucleotides, in some embodiments, between 4-200, e.g., 10-150 nucleotides.

“Clonal” copies of a polynucleotide means the copies are identical in sequence. In some embodiments, there are at least 100, 1000, 10⁴ or more clonal copies of oligonucleotides linked to a bead.

The term “amplification reaction” refers to any in vitro means for multiplying the copies of a target sequence of nucleic acid in a linear or exponential manner. Such methods include but are not limited to polymerase chain reaction (PCR); DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3 SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

The term “amplification reaction mixture” refers to an aqueous solution comprising the various reagents used to amplify a target nucleic acid. These include enzymes, aqueous buffers, salts, amplification primers, target nucleic acid, and nucleoside triphosphates. Amplification reaction mixtures may also further include stabilizers and other additives to optimize efficiency and specificity. Depending upon the context, the mixture can be either a complete or incomplete amplification reaction mixture

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-30 nucleotides, in length. The length and sequences of primers for use in PCR can be designed based on principles known to those of skill in the art, see, e.g., Innis et al., supra. Primers can be DNA, RNA, or a chimera of DNA and RNA portions. In some cases, primers can include one or more modified or non-natural nucleotide bases. In some cases, primers are labeled.

A nucleic acid, or a portion thereof, “hybridizes” to another nucleic acid under conditions such that non-specific hybridization is minimal at a defined temperature in a physiological buffer (e.g., pH 6-9, 25-150 mM chloride salt). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 65, 70, 75, or 80° C. In some embodiments, the defined temperature at which specific hybridization occurs is 37, 40, 42, 45, 50, 55, 60, 65, 70, 75, or 80° C.

A “template” refers to a polynucleotide sequence that comprises the polynucleotide to be amplified, flanked by or a pair of primer hybridization sites. Thus, a “target template” comprises the target polynucleotide sequence adjacent to at least one hybridization site for a primer. In some cases, a “target template” comprises the target polynucleotide sequence flanked by a hybridization site for a “forward” primer and a “reverse” primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanosine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

Polymerases include both DNA-dependent polymerases and RNA-dependent polymerases such as reverse transcriptase. At least five families of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil). Prior to partitioning a solution, the solution is referred to as a “bulk” solution.

As used herein a “barcode” is a short nucleotide sequence (e.g., at least about 4, 6, 8, 12, 15, 20, 50 or 75 or 100 nucleotides long or more) that identifies a molecule to which it is conjugated or from the partition in which it originated. Barcodes can be used, e.g., to identify molecules originating in a partition, bead, or spot as later sequenced from a bulk reaction. Such a barcode can be unique for that partition, bead or spot as compared to barcodes present in other partitions, bead or spot. For example, partitions containing target RNA from single-cells can be subject to reverse transcription conditions using primers that contain different partition-specific barcode sequence in each partition, thus incorporating a copy of a unique “cellular barcode” (because different cells are in different partitions and each partition has unique partition-specific barcodes) into the reverse transcribed nucleic acids of each partition. Thus, nucleic acid from each cell can be distinguished from nucleic acid of other cells due to the unique “cellular barcode.” In some embodiments described herein, barcodes described herein uniquely identify the molecule to which it is conjugated, i.e., the barcode acts as a unique molecular identifier (UMI). The length of the underlying barcode sequence determines how many unique samples can be differentiated. For example, a 1 nucleotide barcode can differentiate 4, or fewer depending on degeneracy, different partitions; a 4 nucleotide barcode can differentiate 4⁴ or 256 partitions or less; a 6 nucleotide barcode can differentiate 4096 different partitions or less; and an 8 nucleotide barcode can index 65,536 different partitions or less.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which are described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### DETAILED DESCRIPTION OF THE INVENTION

It has been determined that one can use non-specific nucleic acid binding proteins to coat certain polynucleotides in various manipulations of nucleic acid solutions (for example, but not limited to partitioning and hybridizations), which can improve specificity of reactions and reduce undesired aggregation of components of molecular biology solutions. This technology can be applied to bulk and compartmentalized reactions, such as those performed in tubes, droplets or in microwells. Additionally, improving homologous interactions with the help of non-specific nucleic acid binding proteins can increase sensitivity and efficiency of oligonucleotide-based detection assays as well as PCR.

Without intending to limit the scope of the invention, it is believed that including non-sequence-specific nucleic acid binding proteins (e.g., recombination and DNA repair DNA/RNA binding proteins) in a solution with nucleic acids decreases non-specific intra- and intermolecular interactions of oligonucleotides, in solution or linked on beads, and can be used to increase target-specific sequences' capture via hybridization by the oligonucleotides. Inclusion of the non-specific nucleic acid binding proteins in solutions with beads also reduce aggregation of the beads. As oligonucleotide-linked beads are commonly used to deliver oligonucleotides into partitions, inclusion of non-sequence-specific nucleic acid binding proteins with the oligonucleotide-linked beads allows for improved partitioning by for example reducing aggregation which can otherwise lead to higher levels of multiple beads within one partition.

A number of non-sequence-specific nucleic acid binding proteins are known and can be used in the methods and compositions described herein. “Non-sequence specific” in this context refers to the ability of the proteins to bind to nucleic acid independent of the nucleic acid sequence. While some variation of binding may occur between sequences, in general a non-sequence-specific binding protein will bind to many different nucleic acid sequences with substantially similar affinity. This is in contrast to, for example, transcription factors or sequence-specific restriction enzymes, that target a particular sequence or small number of sequences with a considerably strong affinity (e.g. 100-fold or more greater) than other non-target sequences. A benefit of a non-sequence-specific nucleic acid binding protein is that many or most sequences within a nucleic acid molecule will be bound to some degree such that the effects described herein occur in a measurable manner.

In some embodiments, the non-sequence-specific nucleic acid binding protein is selected from a nucleic acid (e.g., DNA) recombination protein and/or a DNA repair protein. Exemplary non-specific nucleic acid binding protein can include, for example, RecA, RecO, RecN, RadA, Rad51, Rad52 and UvsX. It will be appreciated that orthologous proteins of each of these proteins can be identified from a number of species and be used as described herein.

Exemplary RecA proteins can include, but are not limited to E. coli or Thermus aquaticus RecA, for example as described in Uniprot accession number P0A7G6 or P48296, respectively or RecA proteins substantially identical to

(SEQ ID NO: 1)
MAIDENKQKALAAALGQIEKQFGKGSIMRLGEDRSMDVETISTGSLSLDI
ALGAGGLPMGRIVEIYGPESSGKTTLTLQVIAAAQREGKTCAFIDAEHAL
DPIYARKLGVDIDNLLCSQPDTGEQALEICDALARSGAVDVIVVDSVAAL
TPKAEIEGEIGDSHMGLAARMMSQAMRKLAGNLKQSNTLLIFINQIRMKI
GVMFGNPETTTGGNALKFYASVRLDIRRIGAVKEGENVVGSETRVKVVKN
KIAAPFKQAEFQILYGEGINFYGELVDLGVKEKLIEKAGAWYSYKGEKIG
QGKANATAWLKDNPETAKEIEKKVRELLLSNPNSTPDFSVDDSEGVAETN
EDF
or
(SEQ ID NO: 2)
MEENKRKSLE NALKTIEKEF GKGAVMRLGE MPKLQVDVIP
TGSLGLDLAL GIGGIPRGRV TEIFGPESGG KTTLALTIIA
QAQKGGGVAA FVDAEHALDP LYAKKLGVDV QELLVSQPDT
GEQALEIVEL LARSGAVDVI VVDSVAALVP KAEIEGEMGD
QHVGLQARLM SQALRKLTAV LSKSNTAAIF INQVREKVGV
MYGNPETTPG GRALKFYSSV RLDVRKSGQP IKVGNEAVGI
KVKVKVVKNK LAPPFREAEL EIYFGRGLDP VMDLVNVAVA
AGVIEKAGSW FSYGEHRLGQ.
See also, Del Val, et al. Biochem Soc Trans. 2019
Oct. 31;47(5):1511-153.

Exemplary RecO proteins can include, but are not limited to E. coli RecO, for example as described in Uniprot accession number P0A7H3, or RecO proteins substantially identical to

(SEQ ID NO: 3)
MEGWQRAFVLHSRPWSETSLMLDVFTEESGRVRLVAKGARSKRSTLKGAL
QPFTPLLLRFGGRGEVKTLRSAEAVSLALPLSGITLYSGLYINELLSRVL
EYETRFSELFFDYLHCIQSLAGVTGTPEPALRRFELALLGHLGYGVNFTH
CAGSGEPVDDTMTYRYREEKGFIASVVIDNKTFTGRQLKALNAREFPDAD
TLRAAKRFTRMALKPYLGGKPLKSRELFRQFMPKRTVKTHYE.

Exemplary Rad51 proteins can include, but are not limited to human Rad51, for example as described in Uniprot accession number Q06609, or Rad51 proteins substantially identical to

(SEQ ID NO: 4)
GSHMAMQMQL EANADTSVEE ESFGPOPISR LEQCGINAND
VKKLEEAGFH TVEAVAYAPK KELINIKGIS EAKADKILAE
AAKLVPMGFT TATEFHQRRS EIIQITTGSK ELDKLLQGGI
ETGSITEMFG EFRTGKTQIC HTLAVTCQLP IDRGGGEGKA
MYIDTEGTFR PERLLAVAER YGLSGSDVLD NVAYARAFNT
DHQTQLLYQA SAMMVESRYA LLIVDSATAL YRTDYSGRGE
LSARQMHLAR FLRMLLRLAD EFGVAVVITN QVVAQVDGAA
MFAADPKKPI GGNIIAHAST TRLYLRKGRG ETRICKIYDS
PCLPEAEAMF AINADGVGDA KD.

Exemplary Rad52 proteins can include, but are not limited to human Rad52, for example as described in Uniprot accession number P43351, or Rad52 proteins substantially identical to

(SEQ ID NO: 5)
MSGTEEAILGGRDSHPAAGGGSVLCFGQCQYTAEEYQAIQKALRQRLGPE
YISSRMAGGGQKVCYIEGHRVINLANEMFGYNGWAHSITQQNVDFVDLNN
GKFYVGVCAFVRVQLKDGSYHEDVGYGVSEGLKSKALSLEKARKEAVTDG
LKRALRSFGNALGNCILDKDYLRSLNKLPRQLPLEVDLTKAKRQDLEPSV
EEARYNSCRPNMALGHPQLQQVTSPSRPSHAVIPADQDCSSRSLSSSAVE
SEATHQRKLRQKQLQQQFRERMEKQQVRVSTPSAEKSEAAPPAPPVTHST
PVTVSEPLLEKDFLAGVTQELIKTLEDNSEKWAVTPDAGDGVVKPSSRAD
PAQTSDTLALNNQMVTQNRTPHSVCHQKPQAKSGSWDLQTYSADQRTTGN
WESHRKSQDMKKRKYDPS. See also, McDevitt, et al.,
Nature Communications volume 9, Article number:
1091 (2018).

Exemplary UvsX proteins can include, but are not limited to for example Bacteriophage T4 UvsX as described in Uniprot accession number P04529, or UvsX proteins substantially identical to

(SEQ ID NO: 6)
MSDLKSRLIKASTSKLTAELTASKFFNEKDVVRTKIPMMNIALSGEITGG
MQSGLLILAGPSKSFKSNFGLTMVSSYMRQYPDAVCLFYDSEFGITPAYL
RSMGVDPERVIHTPVQSLEQLRIDMVNQLDAIERGEKVVVFIDSLGNLAS
KKETEDALNEKVVSDMTRAKTMKSLFRIVTPYFSTKNIPCIAINHTYETQ
EMFSKTVMGGGTGPMYSADTVFIIGKRQIKDGSDLQGYQFVLNVEKSRTV
KEKSKFFIDVKFDGGIDPYSGLLDMALELGFVVKPKNGWYAREFLDEETG
EMIREEKSWRAKDTNCTTFWGPLFKHQPFRDAIKRAYQLGAIDSNEIVEA
EVDELINSKVEKFKSPESKSKSAADLETDLEQLSDMEEFNE.

In some embodiments, two or more different (of different amino acid sequence) non-sequence-specific nucleic acid proteins can be used. For example, 2, 3, 4, 5 or more different non-sequence-specific nucleic acid proteins can be used in the methods described herein. In some embodiments, use of two or more different non-sequence-specific nucleic acid proteins can allow for improved results.

In some embodiments, one or two or more non-sequence-specific nucleic acid binding proteins are mixed in a solution with a plurality of oligonucleotide-linked beads. This can be performed in a bulk solution, i.e., prior to partitioning the solution, such that the oligonucleotides of the oligonucleotide-linked beads, are bound by the non-sequence-specific nucleic acid binding proteins, decreasing or preventing aggregation between the beads.

Exemplary oligonucleotide-linked beads include for example hydrogel beads. Beads linked to clonal copies of oligonucleotides are commonly used to deliver bead-specific (e.g., partition-specific) barcodes, as well as other barcodes, into partitions. Various methods are available for inserting beads into partitions, depending for example of the type of partition. However, generally, one goal of insertion of oligonucleotide-linked beads into partitions is to achieve single beads per partition. However, oligonucleotide-linked beads can have some affinity for each other, leading to aggregation of the beads, resulting in delivery of aggregates of beads into some partitions rather than single bead delivery. By initially combining a mixture of oligonucleotide-linked beads with the non-sequence-specific nucleic acid binding proteins, aggregation of the oligonucleotide-linked beads will be reduced allowing for delivery of the beads into the partitions.

Hydrogel beads can be composed of various materials. Exemplary hydrogel beads can be, for example, agarose hydrogel beads or polyacrylamide hydrogel beads. In other embodiments, the hydrogels comprise, for example, polystyrene, silica, polymethylmethacrylate, alginate, poly ethylene glycol (PEG), nylon, and/or other polymers, with or without crosslinking. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240. Beads can be magnetic or paramagnetic as desired.

Oligonucleotides can be linked to beads as desired. The solid support surface of the bead can be modified to include a linker for attaching barcode oligonucleotides. The linkers may comprise a cleavable moiety. Non-limiting examples of cleavable moieties include a disulfide bond, a deoxyuridine moiety, and a restriction enzyme recognition site. Methods of linking oligonucleotides to beads are described in, e.g., WO 2015/200541. In some embodiments, the oligonucleotide configured to link a hydrogel bead to the barcode is covalently linked to the hydrogel. Numerous methods for covalently linking an oligonucleotide to one or more hydrogel matrices are known in the art. As but one example, aldehyde derivatized agarose can be covalently linked to a 5′-amine group of a synthetic oligonucleotide.

Distributing the oligonucleotide-linked beads, bound by non-sequence-specific nucleic acid binding proteins, into partitions can be achieved by any methods available. For example, partitions can be pre-formed, optionally with other agents and optionally target nucleic acids from a biological sample and the beads and non-covalently linked oligonucleotides can be injected or otherwise introduced into the partitions. Methods and compositions for delivering reagents to one or more partitions include microfluidic methods as known in the art; droplet or microcapsule merging, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

In other embodiments, for example in which the partitions are droplets, one can form droplets as an emulsion with an immiscible fluid such as oil such that the bulk solution forms droplets that contain the beads and non-covalently linked oligonucleotides, optionally with other reagents and/or a sample nucleic acid. Methods of emulsion formation are described, for example, in published patent applications WO 2011/109546 and WO 2012/061444,

Distribution of beads into partitions (e.g., such as droplets) can be dictated by a Poisson distribution, in some embodiments. Depending on the end use, the average number of beads per partition can be less than 1 (e.g., 0.2-0.9), 1, or more than 1 (e.g., 1-3 or more, e.g., fewer than 5). In some embodiments, it is desirable to avoid multiple beads in a partition and in these cases many partitions may be left empty such that a majority of partitions that contain a bead only contain one bead. In other embodiments, e.g., in which deconvolution methods can be used to decipher sequencing results where multiple beads occur in a single partition, more beads can be loaded on average per partition, with deconvolution being used after to resolve sequencing results. See, e.g., PCT/US2017/012618; PCT/US2019/015638; PCT/US2020/36699. Nevertheless, there will be a benefit if aggregation of the beads is decreased even if more than one bead is inserted into a partition.

Any type of partitions can be used with the methods and compositions described herein. In some embodiments, the beads can be inserted into partitions (e.g., droplets or wells). In some embodiments, the beads are encapsulated into aqueous droplets in a water-in-oil emulsion. Methods and compositions for partitioning a sample are described, for example, in published patent applications WO 2010/036,352, US 2010/0173,394, US 2011/0092,373, and US 2011/0092,376, the contents of each of which are incorporated herein by reference in the entirety. The plurality of mixture partitions can be in a plurality of emulsion droplets, or a plurality of wells, etc. The partitions can be picowells, nanowells, or microwells. In some embodiments, there are at least e.g., 100,000 wells, or 200,000 wells e.g., 100,000-500,000 wells. Exemplary wells can have a volume capacity of e.g., 10-50 picoliters. Exemplary wells include those as described in U.S. Patent Publication No. US2021/0283608. The mixture partitions can be pico-, nano-, or micro-reaction chambers, such as pico, nano, or microcapsules. The mixture partitions can be pico-, nano-, or micro-channels. The mixture partitions can be droplets, e.g., emulsion droplets.

In addition to improving distribution of beads into partitions by reducing aggregation, a separate advantageous use of non-sequence-specific nucleic acid binding proteins is to improve nucleic specific nucleic acid hybridization and reduction of non-specific hybridization. Thus, non-sequence-specific nucleic acid binding proteins can be included in any nucleic acid reaction mixture to increase sensitivity and efficiency of oligonucleotide-based hybridization. Thus, for example, any type of nucleic acid hybridization assay, including but not limited to nucleic acid amplification methods, including but not limited to PCR, can be improved by inclusion of a sufficient amount of the non-specific nucleic acid binding proteins in the reaction mixture of the method.

Thus in some embodiments, non-sequence-specific nucleic acid binding proteins are included in a mixture comprising an oligonucleotide and a target nucleic acid under conditions to induce hybridization and then detection of specific hybridization between the oligonucleotide and the target nucleic acid (if present). The non-specific nucleic acid binding proteins can be included in an amount sufficient to improve specificity of complementary nucleic acids, e.g., the oligonucleotide and the target nucleic acid, optionally by reducing non-sequence-specific nucleic acid interactions, e.g., nucleic acids that are not 100% complementary. Improved specificity of hybridization of complementary nucleic acids can be assayed as desired. In one embodiments, specificity can be assayed by hybridizing an oligonucleotide linked to a bead to a mixture of fully and partially complementary nucleic acids in the presence and absence of the non-sequence-specific nucleic acid binding protein(s), removing non-hybridizing nucleic acids from the bead, and detecting the amount of partially complementary nucleic acids that remain associated with the beads. This can involve, for example, pelleting the beads in the hybridization reaction, washing away non-hybridizing nucleic acids, then eluting hybridizing nucleic acids and assaying the amount of non-fully complementary nucleic acids, e.g., via qPCR. Inclusion of the non-sequence-specific nucleic acid binding proteins in a sufficient amount will result in less hybridization of non-fully complementary nucleic acids compared to a control otherwise identical but lacking the non-sequence-specific nucleic acid binding proteins.

Any format for detection of nucleic a hybridization can be used. In some embodiments target nucleic acids can be amplified, optionally in partitions, and then the amplified target nucleic acids can be detected, preferably such that the presence or absence of amplified product can be detected optically. Amplification can be performed under isothermal or thermocyclic conditions. For example, the amplification can compromise reverse transcription (RT), RT-PCR, Loop-mediated isothermal amplification (LAMP) (which is isothermal), RT-LAMP, or bridge amplification.

A target nucleic acid can be any nucleic acid as desired. For example, the nucleic acid can be a target nucleic acid from a biological sample. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, stool, urine, etc. In some embodiments, the sample is a sample comprising cells. In some embodiments, the sample is a single-cell sample.

In some embodiments, the target nucleic acid is RNA. RNA can reverse transcribed by adding the appropriate reverse transcription regents (e.g., a reverse transcriptase, nucleotides, one or more primer, which optionally is a primer comprising a polyT 3′ end) along with a sufficient amount of non-sequence-specific nucleic acid binding proteins as described herein, and extending the primer using the RNA as a template to form first strand cDNA molecules. As a further possible step, the first strand cDNA can be converted to double stranded cDNA through second strand synthesis (e.g., by providing appropriate reagents, e.g., an appropriate primer and DNA polymerase).

Optionally, the hybridization or detection of the hybridization or both, can occur in partitions, allowing for example for “digital” signals, wherein each partition produces a detectable signal if a oligonucleotide probe or primer hybridizes with a target nucleic acid in the partition. Quantification of the hybridization can be determined by quantifying the number of signal-generating partitions, optionally as a ratio of total number of partitions, or total number containing an oligonucleotide primer or probe.

In other embodiments, the inclusion of one or more non-sequence specific nucleic acid binding protein in a hybridization in a microchannel can improve specificity and reduce aggregation where a target nucleic acid is hybridized to an oligonucleotide linked to an immobilized bead. Thus, in some embodiments, sample nucleic acids are flowed through a microfluidic channel and passed an immobilized bead linked to an oligonucleotide under conditions in which a complementary target nucleic acid, if present, hybridizes to the oligonucleotide linked to the bead. As noted above, the inclusion of a sufficient amount of one or more non-sequence specific nucleic acid binding protein in the microfluidic channel will improve specificity and reduce aggregation. The bead can be immobilized to the microfluidic channel as desired. In some embodiments, the bead is reversibly immobilized such that the bead, linked oligonucleotide and any hybridized target nucleic acid can be released following a wash, allowing for collection and/or detection of the presence, absence or amount of hybridized target nucleic acid at a second location in the microfluidic channel. In some embodiments, the bead is immobilized in an electrical field. For example in some embodiments, the bead is immobilized using optical tweezers. Optical tweezer technology is summarized for example in Bustamante et al., Nature Reviews Methods Primers volume 1, Article number: 25 (2021).

Detection of the target molecule, which may be amplified, can be performed in any way useful to generate a detectable signal. Preferably the signal can be detected optically, for example such that an automated scanner can detect the presence, absence or quantity of signal in each well. Signal can increase upon presence of the target molecule or in other embodiments signal can decrease (e.g., in the case of quenching of signal in the presence of the target molecule).

In some embodiments, the reaction mixture where hybridization occurs, which can optionally be a partition, comprises one or more optically detectable agents such as a fluorescent agent, phosphorescent agent, chemiluminescent agent, etc. Numerous agents (e.g., dyes, probes, or indicators) are known in the art and can be used. See, e.g., Invitrogen, The Handbook—A Guide to Fluorescent Probes and Labeling Technologies, Tenth Edition (2005). Fluorescent agents can include a variety of organic and/or inorganic small molecules or a variety of fluorescent proteins and derivatives thereof. In some embodiments, the agent is a fluorophore. A vast array of fluorophores are reported in the literature and many are readily available from commercial suppliers to the biotechnology industry. Literature sources for fluorophores include Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995). Non-limiting examples of fluorophores include cyanines, fluoresceins (e.g., 5′-carboxyfluorescein (FAM), Oregon Green, and Alexa 488), HEX, rhodamines (e.g., N,N,N′,N′-tetramethyl-6-carboxyrhodamine (TAMRA), tetramethyl rhodamine, and tetramethyl rhodamine isothiocyanate (TRITC)), eosin, coumarins, pyrenes, tetrapyrroles, arylmethines, oxazines, polymer dots, and quantum dots.

In some embodiments, the detectable agent is an intercalating agent. Intercalating agents produce a signal when intercalated in double stranded nucleic acids. Exemplary intercalating agents include e.g., 9-aminoacridine, ethidium bromide, a phenanthridine dye, EvaGreen, PICO GREEN (P-7581, Molecular Probes), EB (E-8751, Sigma), propidium iodide (P-4170, Sigma), Acridine orange (A-6014, Sigma), thiazole orange, oxazole yellow, 7-aminoactinomycin D (A-1310, Molecular Probes), cyanine dyes (e.g., TOTO, YOYO, BOBO, and POPO), SYTO, SYBR Green I (U.S. Pat. No. 5,436,134: N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR Green II (U.S. Pat. No. 5,658,751), SYBR DX, OliGreen, CyQuant GR, SYTOX Green, SYTO9, SYT010, SYT017, SYBR14, FUN-1, DEAD Red, Hexidium Iodide, ethidium bromide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, LDS 751 (U.S. Pat. No. 6,210,885), and the dyes described in dyes described in Georghiou, Photochemistry and Photobiology, 26:59-68, Pergamon Press (1977); Kubota, et al., Biophys. Chem., 6:279-284 (1977); Genest, et al., Nuc. Ac. Res., 13:2603-2615 (1985); Asseline, EMBO J., 3: 795-800 (1984); Richardson, et. al., U.S. Pat. No. 4,257,774; and Letsinger, et. al., U.S. Pat. No. 4,547,569.

One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the TaqMan™ probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Thus, in some embodiments, the detectable agent is a molecular beacon oligonucleotide probe. The “beacon probe” method relies on the use of energy transfer. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched.

In some embodiments, the sample nucleic acids can be detected using the SHERLOCK method (Specific High Sensitivity Enzymatic Reporter UnLOCKing). This method provides an in vitro nucleic acid detection platform with high (or single-molecule) sensitivity based on nucleic acid amplification and collateral cleavage of a reporter ssDNA, allowing for real-time detection of the target. Methods of using CRISPR in SHERLOCK are described in detail, e.g., in Gootenberg, et al. “Nucleic acid detection with CRISPR-Cas13a/C2c2,” Science, 356(6336):438-442 (2017) and US Patent Publication No. 20180340219.

In some embodiments, hybridization between one or more oligonucleotide and a target nucleic, in the presence of a sufficient amount of a non-sequence specific nucleic acid binding protein, occurs in an amplification, which resulting amplicon is later detected by nucleotide sequencing, e.g., next generation sequencing. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/0841,32, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of introducing oligonucleotide-linked beads into partitions, the method comprising,
providing a bulk solution comprising (i) a plurality of the oligonucleotide-linked beads and (ii) an amount of a non-sequence specific nucleic acid binding protein sufficient to reduce aggregation by the beads; and
introducing the beads into the partitions such that the number of beads in the partitions averages less than 5 beads per partition.

2. The method of claim 1, wherein the partitions are microwells or droplets.

3. The method of claim 1, wherein at least a majority of the oligonucleotides coated on a bead have identical sequences and the oligonucleotides on different beads can be distinguished by a barcode sequence on the oligonucleotide.

4. The method of any one of claims 1-3, wherein the oligonucleotides comprise a free 3′ end that comprises at least 6 contiguous thymine nucleotides that form a poly-T sequence.

5. The method of any one of claims 1-4, wherein the partitions comprise single cells per partition during the introducing.

6. The method of claim 5, wherein, following the introducing, lysing the cells in the partitions and hybridizing the oligonucleotides in the partitions to target nucleic acids from the cells.

7. The method of claim 5, further comprising, before the hybridizing, adding a further amount of the non-sequence specific nucleic acid binding protein to the partitions.

8. The method of claim 6, wherein the oligonucleotides are linked to the beads prior to the hybridizing.

9. The method of claim 6, wherein the oligonucleotides are released from the beads prior to the hybridizing.

10. The method of claim 6, further comprising extending the oligonucleotides with a polymerase in a template-specific manner to link a reverse complement of the target nucleic acids to the oligonucleotides.

11. The method of claim 6, wherein the nucleic acids are RNA.

12. The method of claim 6, wherein the nucleic acids are DNA.

13. The method of any one of claims 1-12, wherein the non-sequence specific nucleic acid binding protein is selected from the group consisting of RecA, RecO, RecN, RadA, Rad51, Rad52 and UvsX.

14. A solution comprising (i) a plurality of the oligonucleotide-linked beads and (ii) an amount of a non-sequence specific nucleic acid binding protein sufficient to reduce aggregation by the beads.

15. A method of introducing oligonucleotide-linked beads into partitions, the method comprising,
providing a plurality of partitions, wherein the partitions comprise (i) an oligonucleotide-linked bead and (ii) a non-sequence specific nucleic acid binding protein, and (iii) a target nucleic acid; and
hybridizing the oligonucleotide to the target nucleic acid in the presence of the non-sequence specific nucleic acid binding protein.

16. The method of claim 15, wherein the method comprises releasing the oligonucleotides from the bead prior to the hybridizing.

17. The method of claim 15, wherein at least a majority of the oligonucleotides linked to a bead have identical sequences and the oligonucleotides on different beads can be distinguished by a barcode sequence on the oligonucleotide.

18. The method of claim 15, wherein the partitions are microwells or droplets.

19. The method of any one of claims 15-18, wherein the non-sequence specific nucleic acid binding protein is selected from the group consisting of RecA, RecO, RecN, RadA, Rad51, Rad52 and UvsX.

20. A method of detecting a nucleic acid in a microfluidic channel, the method comprising,
flowing a nucleic acid sample in a microfluidic channel passed an immobilized oligonucleotide-linked bead in the presence of a non-sequence specific nucleic acid binding protein under conditions sufficient to allow for specific hybridization of the oligonucleotide to a target nucleic acid in the nucleic acid sample; and
detecting hybridization of the target nucleic acid to the oligonucleotide.
